(19) United States
(12) Patent Application Publication
(10) Pub. No.: US 2011/0144493 A1
(43) Pub. Date: Jun. 16, 2011

(54) ULTRASOUND DIAGNOSTIC AND THERAPEUTIC DEVICES

(75) Inventor: Armen P. Sarvazyan, Lambertville, NJ (US)

(73) Assignee: ARTANN LABORATORIES, INC., Trenton, NJ (US)

(21) Appl. No.: 13/028,301

(22) Filed: Feb. 16, 2011

Related U.S. Application Data
(63) Continuation-in-part of application No. 12/766,383, filed on Apr. 23, 2010.
Application No. 12/766,383 is a continuation-in-part of application No. 11/223,259, filed on Sep. 10, 2005, now Pat. No. 7,713,200.

(51) Int. Cl. A61N 7/00; A61B 8/00

(52) U.S. Cl. 600/439

(57) ABSTRACT

An ultrasound diagnostic and therapeutic device includes a catheter equipped with an intracorporeal ultrasound transducer at its tip, an extracorporeal ultrasound transmitter, an imaging electronic unit and a time-reversal electronic unit. The intracorporeal transducer may be used to record an image of surrounding tissues so as to identify a treatment site. The same transducer is then used as a beacon to receive an ultrasound impulse from the extracorporeal transmitter. The impulse response signal from the intracorporeal transducer is then time-reversed so that high-intensity focused ultrasound can be generated at the location of the intracorporeal transducer. The device is capable of shaping the area of focused ultrasound to correspond to that of the treatment site.

### REFERENCE TO GOVERNMENT-SPONSORED RESEARCH

This invention was made with the U.S. government support under SBIR grant No. NS065524 entitled “Time-reversal acoustic device for enhanced drug delivery for brain gliomas” and awarded by the National Institute of Health, National Institute of Neurological Disorders and Stroke. The government has certain rights in this invention.

### CROSS-REFERENCE DATA

This patent application is a continuation-in-part of a co-pending U.S. patent application No. 12/766,383 filed 23 Apr. 2010 entitled “Ultrasound-assisted drug-delivery method and system based on time reversal acoustics”, which in turn is a continuation-in-part of U.S. patent application No. 11/223,259 filed 10 Sep. 2005 entitled “Wireless beacon for time-reversal acoustics, method of use and instrument containing thereof”, now issued as U.S. Pat. No. 7,713,200. All of the above mentioned patent documents are incorporated herein by reference in their respective entireties.

### BACKGROUND

The present invention relates generally to medical devices and methods. More particularly, the invention relates to ultrasonic diagnostic and therapeutic devices based on Time-Reversal Acoustics (TRA) principles for focusing ultrasound as well as combined use of the extracorporeal and intracorporeal ultrasonic transducers.

Focusing of ultrasonic waves is a fundamental aspect of most medical applications of ultrasound. The efficiency of ultrasound focusing in biological tissues is often significantly limited by spatial heterogeneities of sound velocity in tissues and the presence of various reflective surfaces and boundaries. The refraction, reflection and scattering of ultrasound waves in inhomogeneous media can greatly distort an otherwise focused ultrasound field. There are many methods for improving the ultrasonic focusing in complex media based on the phase and amplitude corrections in focusing system but they often do not provide a necessary improvement. The concept of TRA provides an elegant way of simultaneous temporal and spatial focusing of acoustic energy in such inhomogeneous media. The general concept of TRA is described in a publication authored by M. Fink entitled “Time-reversed acoustics” (Scientific American, Nov. 1999, pp. 91-97), which is incorporated herein by reference. U.S. Pat. No. 5,092,336 by Fink, which is also incorporated herein by reference, describes one example of a device for localization and focusing of acoustic waves in tissues.

An important issue in the TRA method of focusing ultrasound energy is related to obtaining initial impulse response signal from the treatment area. It is necessary to have a beacon to accept the initial ultrasound impulse sent towards the desired focal region and transmit the impulse response signal back to the control system. In the TRA focusing systems described in the prior art, the most commonly used beacon is a hydrophone placed at the selected treatment point. Other examples of beacons described in the art are highly reflective targets that provide an acoustical feedback signal for TRA focusing of an acoustic beam. Several examples of TRA focusing systems employing a passive ultrasound reflector or an active ultrasound emitter as a TRA beacon are described in the U.S. Pat. No. 7,201,749 and in a European Patent Application No. EP1449564 by Govari et al., both are incorporated herein by reference in their entireties.

Ability of TRA focusing systems to focus ultrasound with great precision in a body of a patient has an important implication for various therapeutic applications. Ultrasound- assisted drug delivery and noninvasive ablation utilizing high-intensity focused ultrasound (HIFU) are examples of therapies widely used in treating tumors. HIFU can selectively ablate a targeted tumor at a depth of several centimeters without damaging the surrounding or overlying healthy tissues. Both thermal and cavitational mechanisms of tissue treatment have been employed in ultrasound therapy. Ultrasound-induced cavitation in tissue involves creation and oscillation of gas bubbles. Thermal ablation is currently the most extensively explored technique of ultrasonic treatment of lesions. A focused ultrasound beam causes a rapid temperature rise in tissue to cytotoxic levels within the predefined focal volume. Optimal parameters of HIFU, such as intensity, frequency and duration of pulses, are typically quite different for cavitational and thermal mechanisms employed in a particular type of treatment.

An important aspect of efficient HIFU therapy is the requirement for accurate focusing of ultrasound at the treatment site in the body, such as a tumor. Such treatment site may have a complex three-dimensional shape. Some useful methods of controlling the shape of the focus spot and therefore optimizing the ultrasound exposure are described in the following publications co-authored by the inventor of the present invention: (1) Choi BK, Sutin A, Sarvazyan A. Formation of desired waveform and focus structure by time reversal acoustic focusing system. Proceedings of the 2006 IEEE International Ultrasonics Symposium, Vancouver, Canada, 2006:2177-2181; (2) Sarvazyan A, Fillinger L, Gavrilov L. Time-reversal acoustic focusing system as a virtual random phased array. IEEE Trans Ultrason Ferroelectr Freq Control. 2010 Apr;57(4):812-7; and (3) Sarvazyan AP, Fillinger L, Gavrilov LR. A comparative study of systems used for dynamic focusing of ultrasound. Acoustical Physics 2009; 55(4-5):630-637. All these publications are incorporated herein by reference in their entireties.

HIFU is also shown to be effective in a targeted drug delivery, especially for cancer treatment. Tumor chemotherapy is often associated with severe side effects caused by the interactions of cytotoxic drugs with healthy tissues. In addition, tumor cells often develop resistance to drugs in the course of chemotherapy (cross-resistance or multi-drug resistance). Direct injection of drugs in the tumor substantially reduces or eliminates side effects of chemotherapy and increases therapeutic windows of drugs. Desired drug agents are typically bound to nano- or micro-scale carriers, and administered intravenously to a patient to be then activated by ultrasound. This allows a high dose of toxic drugs to be delivered specifically to a targeted area, while minimizing negative side effects.

A decision to conduct a certain therapeutic procedure is typically made on the basis of finding of tissue abnormality. Diagnosis of such abnormality is frequently done using some form of imaging of tissue. One example of such tissue imaging modalities designed specifically for examining blood vessels and surrounding vessels is intravascular ultrasound (IVUS). As opposed to ultrasound imaging of large internal organs, IVUS requires a high resolution of imaging necessary for visualizing submillimeter size structures of closely-located surrounding tissues and therefore uses high frequency of ultrasound for that purpose. With IVUS, a specially-designed imaging catheter with a miniaturized high-frequency intracorporeal ultrasound transducer attached to or near its distal end is inserted into a blood vessel, such as a coronary artery vessel. The intracorporeal ultrasound transducer is a part of an imaging system used to create a cross-sectional image from within the vessel or organ to allow physicians to see a close-up high-resolution image of surrounding tissues which is helpful in differentiating a diseased state from a healthy state.

As already alluded to above, intravascular imaging is typically conducted using a very high frequency ultrasound to get sufficiently high resolution images. For example, the Atlantis™ SR Plus catheter produced by Boston Scientific (Natick, MA) operates at 40 MHz and Eagle Eye™ catheter produced by Volcano Therapeutics (San Diego, CA) operates at 45 MHz. In contrast, therapeutic HIFU systems typically operate at much lower frequencies in the range from hundreds of kHz to several MHz. Consequently, conventional intracorporeal ultrasound transducers adapted for IVUS purposes cannot be effectively used to deliver high-intensity ultrasound therapy to the detected lesion. However, using the method of the present invention, the intracorporeal transducer of an IVUS catheter can be made to serve as a beacon for accurate focusing of ultrasound over the detected lesion by an extracorporeal TRA focusing system.

Described in the prior art are ultrasonic therapeutic systems based on the use of ultrasound-tipped catheter for delivering acoustic energy at the site of treatment. One example of such system is an EkoSonic System [www.ekoscorp.com] configured for ultrasound-accelerated thrombolysis. This system includes a catheter for selective infusion of a clot-dissolving drug into the occluded vessel. Administration of the drug is followed by sonication for enhancing drug diffusion into the thrombus. A miniature ultrasound transducer mounted at the distal end of the catheter highly limits the possibility of creating ultrasonic fields that are optimally tailored to the geometry of a treatment site. Capability to flexibly change and optimize ultrasonic exposure parameters such as frequency and intensity is also highly limited.

The need therefore exists for catheter-based systems allowing accurate in-vivo tissue diagnosis by providing an image of the tissue and at the same time capable of delivering high-intensity focused therapeutic ultrasound area preferably shaped to correspond to the treatment site.

### SUMMARY

Accordingly, it is an object of the present invention to overcome these and other drawbacks of the prior art by providing a novel diagnostic and therapeutic device configured for tissue imaging and delivery of high-intensity ultrasound.

It is another object of the present invention to provide a system comprising an intracorporeal ultrasound transducer mounted at a tip of a catheter both in generation of an image of surrounding tissues as well as to serve as a beacon for focusing high-intensity ultrasound at the detected lesion.

It is a further object of the invention to provide a device for identifying a treatment site within a body of a patient and delivering a high-intensity ultrasound focused at that treatment site.

It is a further object of the present invention to provide a device configured to initially identify a treatment site, then to conduct the tissue treatment using high-intensity focused ultrasound and finally to monitor the progression of the treatment.

It is yet a further object of the present invention to provide a device for creating a spot of high-intensity ultrasound having a three-dimensional shape corresponding to the shape of detected lesion. This is achieved by using the intracorporeal ultrasound transducer of the catheter as a beacon for the TRA system, capable of receiving initial ultrasound impulses sent by the extracorporeal ultrasound transmitter. A set of received impulse response signals may then be time-reversed and stored in the memory of the TRA electronic unit and paired with data on corresponding locations of the intracorporeal ultrasound transducer. This set of time-reversed impulse response signals can then be used for calculating a focusing signal which is subsequently used to produce a focus area of high intensity ultrasound area having a desired shape.

Additional time-reversed impulse response signals may be theoretically calculated for selected additional points located adjacent to the vicinity of the focusing points where the impulse response is directly recorded, such additional points located both on and near the trajectory of the movement of the catheter tip. These additional impulse response signals may be generated using for example interpolation or extrapolation techniques, details of which may be found in U.S. Patent Application No. 20090270790 and U.S. Patent Application No. 20060241523 incorporated herein by reference in their respective entireties.

Increasing the number of the focusing points for which the impulse response signal is actually recorded or calculated may allow for greater flexibility in creating focal spots having a desired shape tailored to the shape of the lesion that needs to be treated. This may be accomplished by superimposing synchronized time-reversed feedback signals from all selected locations to create a desired focusing signal.

The catheter tip may be optionally retracted away from the treatment site prior to initiation of high-intensity focused ultrasound to prevent interference with high-intensity ultrasound beam. The tissue treatment may be performed with or without additional injection of a drug and/or microbubbles such as ultrasound contrast agent. In the case of treatment by HIFU, the ablation of tissue may be monitored by the imaging system of IVUS after the therapy is delivered.

### BRIEF DESCRIPTION OF THE DRAWINGS

Subject matter is particularly pointed out and distinctly claimed in the concluding portion of the specification. The foregoing and other features of the present disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIG. 1 a schematic diagram showing an embodiment of the device including an intravascular imaging catheter and extracorporeal ultrasound transmitter connected to a TRA electronic unit.

FIG. 2 is an illustration of an external ultrasound transmitter comprised of a reverberator and a plurality of ultrasound transducers mounted in the reverberator cavity

FIG. 3 is a schematic depiction of an integrated diagnostic and therapy protocol according to at least one embodiment of the invention;

FIG. 4 is a schematic depiction of calculating TRA-based focusing signal according to at least one embodiment of the invention;

FIG. 5 is a spatial distribution of the TRA-focused signal in the case of single point focusing (a) and extended focus area for 3-point focusing (b);

FIG. 6 shows another example of producing a composite focus area for ultrasound generated by a sum of four TRA signals; and

FIG. 7 shows examples of formation of complex shapes of a focus area by TRA focusing system such are letters of the alphabet.

### DETAILED DESCRIPTION OF THE PREFERRED EMBODIMENTS OF THE INVENTION

The following description sets forth various examples along with specific details to provide a thorough understanding of claimed subject matter. It will be understood by those skilled in the art, however, that claimed subject matter may be practiced without one or more of the specific details disclosed herein. Further, in some circumstances, well-known methods, procedures, systems, components and/or circuits have not been described in detail in order to avoid unnecessarily obscuring claimed subject matter. In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and make part of this disclosure.

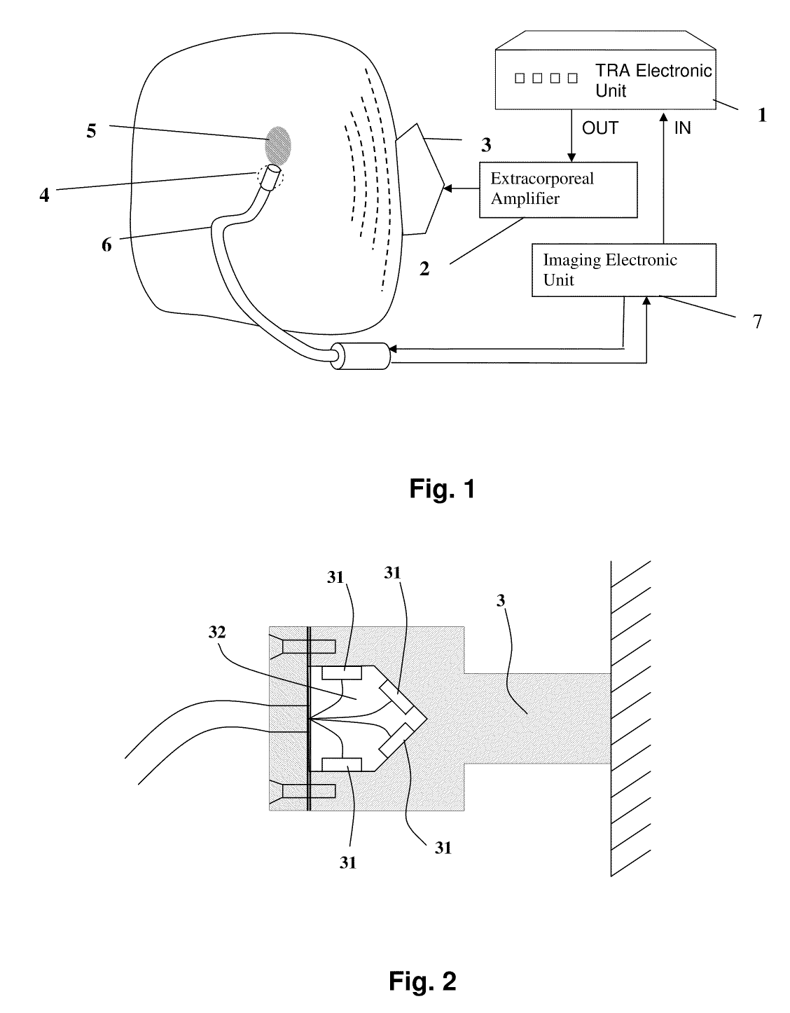

FIG. 1 schematically shows one embodiment of the invention. A TRA electronic unit 1 is shown to be connected through an extracorporeal amplifier 2 to an extracorporeal ultrasound transmitter 3 configured to deliver therapeutic ultrasound pulses into a body of a patient and toward an area of detected lesion 5 near the distal tip 4 of an ultrasound imaging catheter 6.

For the purposes of this description, the term “catheter” includes a variety of medical devices and instruments designed for insertion, penetration or implantation in a body of a patient. Examples of such instruments include catheters, cannulas, tubes, guidewires, probes, needles, trocars, wire leads, etc.

The intracorporeal ultrasound transducer located near or at the end of the distal tip 4 of the catheter 6 may be a standard IVUS transducer or a single broadband transducer or a plurality of broadband transducers sized appropriately for intravascular delivery on a catheter. The IVUS imaging conducted in the pulse-echo mode uses very short high frequency ultrasonic pulses, which is possible only with the use of the broadband ultrasound transducer and receiving electronic circuits. The broadband nature of the intracorporeal ultrasound transducer allows it to be used for two purposes: (a) IVUS imaging of surrounding tissues at high central operating frequencies of 30-60 MHz and (b) receiving of focusing ultrasound impulse at lower therapeutic frequencies of about 0.1-5 MHz to facilitate focusing of HIFU using time-reversal acoustics.

FIG. 2 shows an example of the extracorporeal transmitter consisting of a reverberator 3 and a plurality of ultrasound transducers 31 attached thereto. In this case, transducers 31 are mounted inside the internal cavity 32 of the reverberator 3. The reverberator 3 may be made of material with low attenuation of ultrasound, such as aluminum, to provide long reverberation time of acoustic signal in the body of the reverberator. Longer reverberation is important for the TRA mode of operation because it helps to accumulate more acoustic energy in time.

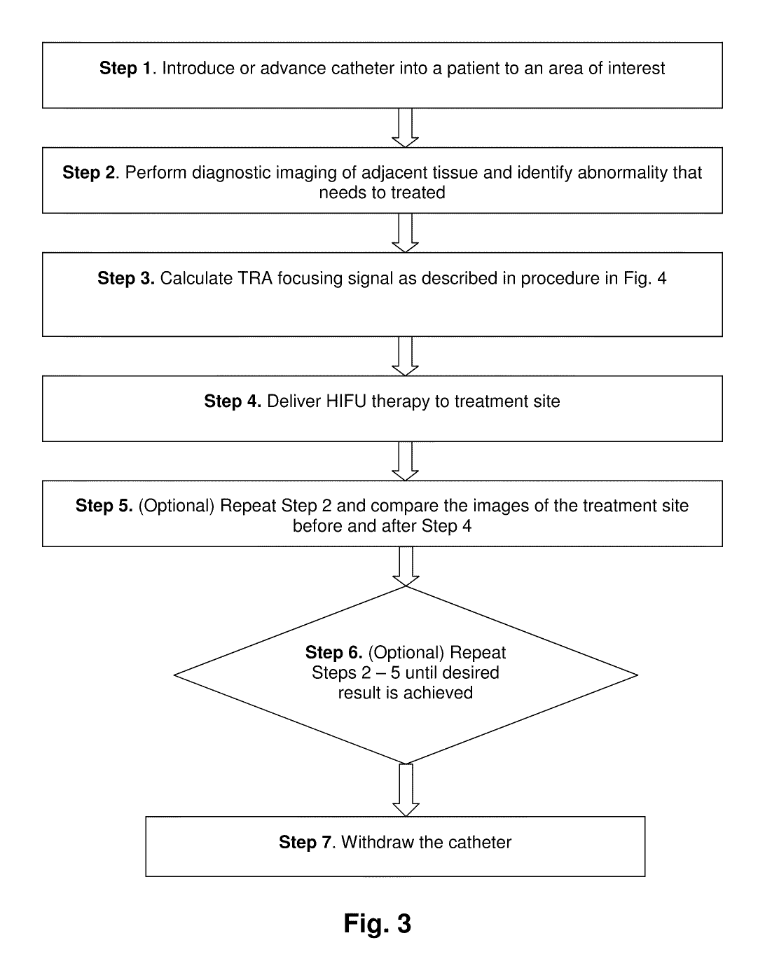

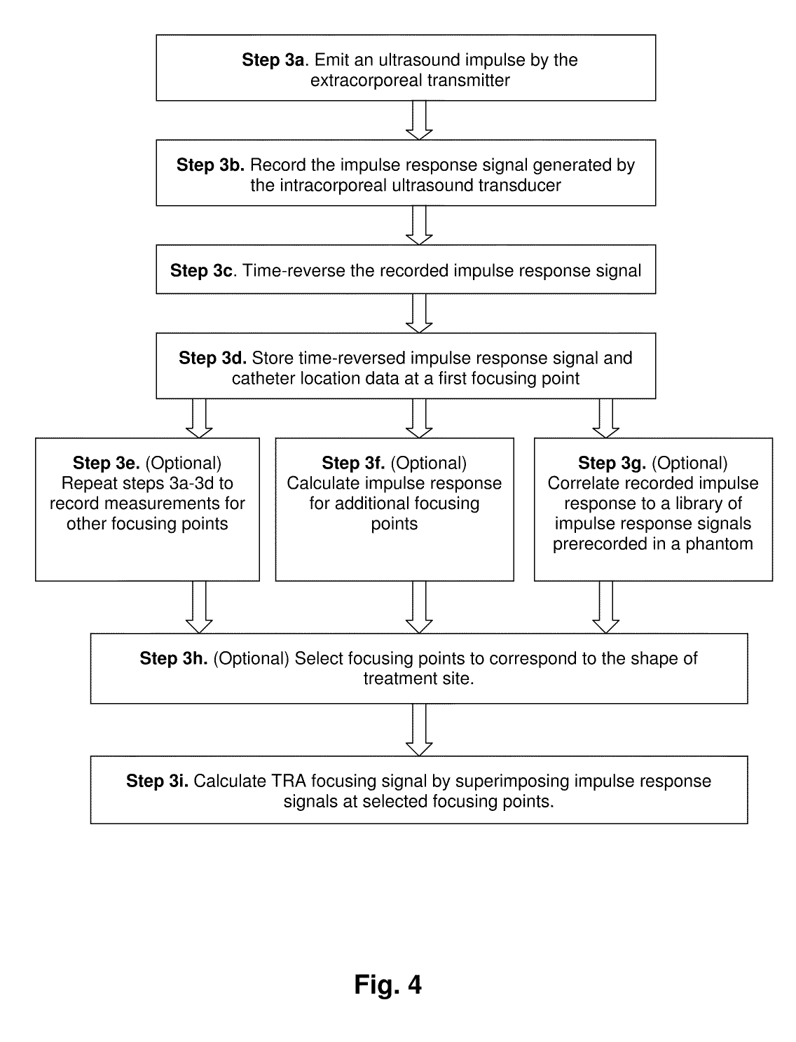

In embodiments, the device of the invention can be operated using principles schematically depicted in FIGS. 3 and 4. The procedure of delivering high-intensity ultrasound using a system described above includes the following steps:

Step 1. Introduce or advance a catheter into a patient to position an intracorporeal ultrasound transducer at an area of interest. Various treatment sites may be located throughout the vasculature or elsewhere in the body. Delivery of the catheter may be done via blood vessels (using arterial or venous vessels) or other small passages in the body such as those of the urinary, gastrointestinal, or respiratory systems. To facilitate transmission of ultrasound in passages not normally filled with fluids, a single or periodic liquid injection at the treatment site may be performed using integrated or separate channels or lumens.

Step 2. Perform diagnostic imaging of surrounding tissue and identify treatment site. This step may be repeated at the same or other locations of the catheter tip. Depending on location and extent of the lesion, multiple images at various locations of the intracorporeal ultrasound transducer may be recorded. As a result, the nature and shape of the treatment site is identified.

Step 3. Calculate the TRA focusing signal as described in procedure in FIG. 4. This step is now described in more detail below and with reference to FIG. 4:

Step 3a. Emit an ultrasound impulse by the extracorporeal ultrasound transmitter. The impulse may be a short burst of ultrasound or may have any appropriate form suitable for a particular therapeutic application.

Step 3b. Record the impulse response signal by the intracorporeal ultrasound transducer at at least a first focusing point. This step may be repeated at various locations along the trajectory of movement of the catheter tip for additional focusing points. Depending on the obtained image of surrounding tissue and other diagnostic factors, not all locations and focusing points may be selected for delivering of focused ultrasound.

Step 3c. Time-reverse the recorded impulse response signal at the first focusing point.

Step 3d. Store time-reversed signal and location data at the first focusing point.

Step 3e (optional). Repeat steps 3a-3d to record measurements for other focusing points. Additional impulse response signals may be recorded at various locations along the trajectory of movement of the catheter tip forming a plurality of focusing points. Location information and time-reversed ultrasound impulse response signal are collected for each focusing point. Position data may be recorded for each additional focusing point in relationship to the first focusing point using any appropriate technique such as tracking motion of the tip as it is moved away from the first focusing point. Having time-reversed impulse response signals for additional focusing points at or adjacent to the trajectory of movement of the intracorporeal ultrasound transducer makes it possible to generate ultrasound focal areas of complex shape optimally tailored to the shape of the treatment lesion. Formation of complex focus spots has an important clinical advantage of tailoring the area of delivering high-intensity ultrasound only to the diseased tissue and sparing healthy tissue from a risk of thermal damage. In case of periodic tissue movement, such as with heart tissue, this procedure may be synchronized with ECG or breathing patterns.

Step 3f (optional). Calculate impulse response for additional focusing points.

Location data for such additional focusing points may also be calculated relative to the first focusing point. The calculated time-reversed impulse response signals for these additional points may be generated using for example interpolation or extrapolation techniques. Details of such techniques may be found in U.S. Patent Application No. 20090270790 and U.S. Patent Application No. 20060241523 incorporated herein by reference in their respective entireties.

Step 3g (optional). Correlate recorded impulse response to a set of impulse responses from library of impulse response signals prerecorded in a phantom fluid having acoustic properties close to those of the measurement site. This step is applicable only in the case when the examined anatomical site is composed of soft tissue away from major skeletal structures. Since velocity of ultrasound in all soft tissues is close to that of saline solution and varies less than 10 percent, an acceptable acoustical phantom of an anatomical site composed of soft tissue could be simply a tank filled with water or saline solution. A reference library may be obtained ahead of time by placing the TRA reverberator in contact with the surface of the saline solution or a body of phantom fluid selected to match the tissue in terms of propagating ultrasound waveforms. A 3D set of impulse response signals in the tank filled with water or saline solution at various coordinates of the recording ultrasound transducer in relationship to the emitting extracorporeal transmitter may be collected. A signal response library is then generated to contain a plurality of response signals and their respective position data. Once the tissue impulse response signal is recorded at a first focusing point, it may be correlated to the library of previously obtained impulse response signals to find the library signal which correlates most closely with the recorded signal. After identification of such signal, its prerecorded coordinates are matched with the location of the first focusing point so the shape of the treatment site can be correlated with the library of prerecorded signals. This step may be used advantageously only in cases when the treatment site is surrounded by soft tissues as the presence of skeletal structures may disturb ultrasound propagation and render the library inaccurate.

Step 3h (optional). Select focusing points to correspond to the shape of treatment site. Once a plurality of focusing points and their corresponding impulse response signals is obtained in steps 3e, 3f, or 3g, some of these focusing signals may be selected to match the shape of the treatment site.

Step 3i. Calculate TRA focusing signal by superimposing impulse response signals at selected focusing points. The focusing signal calculated in this step is calculated to match the shape of the treatment site.

Returning now to FIG. 3, the following describes the rest of the procedure:

Step 4. Deliver HIFU therapy to treatment site. The TRA electronic unit may be activated to cause the extracorporeal ultrasound transmitter to deliver high-intensity focused ultrasound to the treatment site using the focusing signal calculated in step 3.

In embodiments, prior to initiation of the HIFU step, a drug may be injected to the area of the treatment site. The drug may be injected in various forms: as a solution or encapsulated in microbubbles or microparticles adapted for further release thereof as a result of applying HIFU.

In embodiments, the catheter may be partially withdrawn from the treatment site prior to initiation of HIFU to spare the intracorporeal ultrasound transducer from possible damage which may be caused by HIFU. Once HIFU is delivered, the catheter may be returned to its original position so that tissue image may be obtained for assessment of treatment results. Additional treatments may cause repeated partial withdrawals from and returns of the catheter to the treatment site.

Step 5. Repeat Step 2 and compare the images of the treatment site before and after Step 4. This optional step may be conducted to verify success of the treatment.

Step 6. Optionally repeat Steps 1-4 until desired result is achieved. If initial treatment is deemed not sufficient, additional treatments may be delivered to the treatment site.

Step 7. Withdraw the catheter.

When multiple transducers 31 are used to generate HIFU signal as shown in FIG. 2, the impulse response recording procedure described in steps 3a and 3b may be done sequentially and individually by activating each transmitter 31 to send an ultrasound impulse one at a time. To accomplish this, each transducer 31 is individually activated to generate a focusing ultrasound impulse which is recorded by the intracorporeal ultrasound transducer as an impulse response signal and sent back to the TRA electronic unit for time-reversing. Once all transducers 31 have been sequentially activated one at a time and all corresponding impulse response signals are recorded and time-reversed, HIFU therapy may be delivered by synchronously activating all transmitters 31 in a therapy-delivery mode using individually collected impulse response signals.

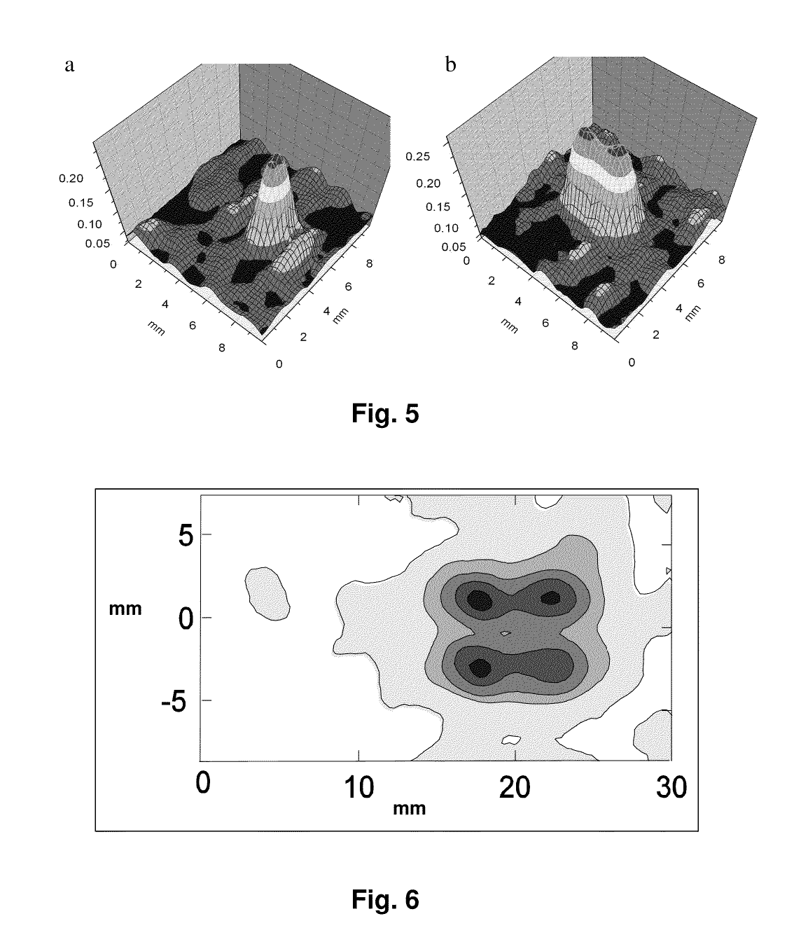

FIG. 5 provides one example of formation of the focus area having a complex shape by superposition of the time-reversed impulse responses separately recorded at several points. Panel A shows a traditional 1-point focus area (seen as a peak on the three-dimensional chart). Panel B shows a more complicated blend formed using 3 separate focusing spots aligned along a straight line. Blending signals using this 3-point focusing spots allows extending the area subjected to HIFU along one desired direction.

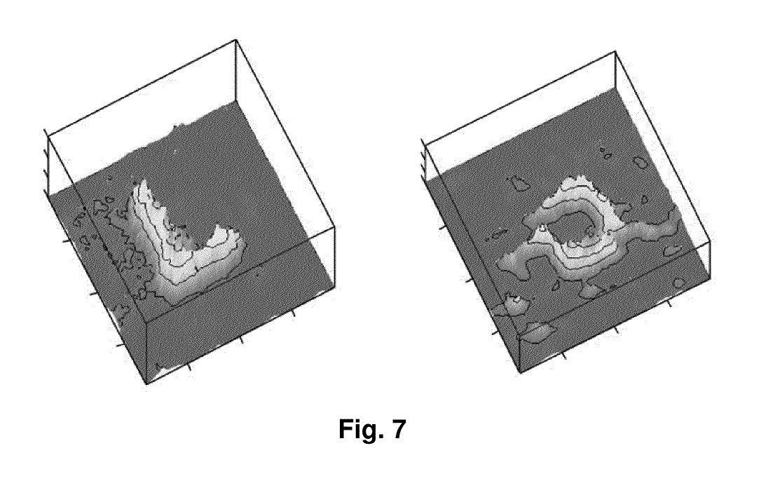

FIG. 6 shows distribution of ultrasound intensity across the plane having 4 focusing spots arranged as corners of a rectangle. Again, blending of the signals from 4 measured locations allows delivering of focused high-intensity ultrasound over a desired area, in this case shaped as a rectangle with well defined corners.

FIG. 7 shows examples of intensity distributions for an even more complex shape of the focus spot. In this case, the focal area was formed using multiple points of focusing and extrapolation of the signals therefrom. As a result, the area was formed to mimic the letters of alphabet, letter L on the left and letter O on the right.

Since the intracorporeal broadband ultrasound transducer of the catheter can detect ultrasound signals at a very wide range of frequencies including those which are much lower than imaging frequencies, one advantage of the present invention is the ability to adjust the frequency of HIFU according to a particular application and treatment mode. The HIFU frequency may be adjusted depending for example on whether cavitation or thermal ablation is required. In all such cases, the range of applicable HIFU frequencies is presumed to be within the operable range of the intracorporeal ultrasound transducer at the catheter tip allowing it to reliably detect the initial ultrasound impulse generated by the extracorporeal transmitter. This advantage may provide for an additional clinical benefit when compared with catheter-mounted transducers configured for delivery of therapeutic ultrasound at a particular fixed frequency.

The following describes advantages of the present invention over conventional ultrasound focusing systems. Such advantages are as follows:

the device of the invention is capable to precisely deliver ultrasound energy to the chosen region regardless of the heterogeneity of the propagation medium, for example to tissues located behind the ribs. The ability to effectively localize ultrasound energy and avoid exposure of surrounding tissues is important in many medical applications including ultrasound ablation therapy and the ultrasound-enhanced drug delivery;

the device of the invention can produce more effective spatial concentration of ultrasound energy than traditional phased array - based systems making it easier to create the focus area having a complex shape tailored to the region that needs to be treated;

the device of the invention can produce pulses with arbitrary desired waveforms in a wide frequency band. Ability to generate various waveforms is important in many applications, for example for optimizing the outcome of the ultrasound- stimulated drug delivery with or without the use of microbubbles where the main mechanism of ultrasound action is related to cavitation and the effectiveness of treatment depends on the frequency and the temporal parameters of the applied signal;

the device of the invention provides much greater flexibility in choosing an optimal frequency for a particular application than conventional phased array-based systems because the TRA focusing is based on multiple reflection of sound waves in a reverberator, a phenomenon which does not depend on frequency. Optimal frequency of ultrasound is different for various mechanisms of therapeutic effects: thermal ablation, stable or transient cavitation or resonance excitation of microbubbles. It may vary in a wide range, from hundreds of kHz to several MHz. For thermal ablation for example, the optimal frequency could be around or above 1 MHz, while for generation of cavitation, lower frequencies could be optimal.

The catheter may include an internal lumen which can be used for delivering microbubbles in the treatment area, such as for example ultrasound contrast agents (UCA). Such agents may make applications of TRA HIFU more efficient, safe, and accurate while producing fewer adverse side effects. Microbubbles may improve energy deposition in a focal area, facilitate a more accurate tailoring of the ablation volume, and help in decreasing required acoustic power and duration of exposure. Another advantageous application of UCA in TRA HIFU therapy is related to ultrasound-enhanced chemotherapy and drug delivery. Microbubbles become active in the ultrasound field by either stable cavitation or inertial cavitation, resulting in the destruction of pathological tissue and/or inducing microstreaming which enhanced the diffusion of drugs through cell membranes for transport of drugs and genes to a specific diseased site.

Examples of procedures in which the present invention may be advantageously used include: intravascular phonophoresis, treatment of restenosis after angioplasty or implantation of a stent, plaque or thrombus ablation / dissolution, dissolution of intravascular blockage, concomitant inhibition of restenosis, inhibition of vascular hyperplasia, inhibition of hyperplasia in vascular fistulas and grafts, neuro analgesia and anesthesia, non-invasive cleaning of the implanted device such as a prosthetic heart valve from undesirable deposits, creating linear lesions for the treatment of atrial fibrillation, selective destruction of vasculature providing nutrients to the tissue, acoustic hemostasis, ablation of blood thrombi, treating of peripheral blood vessel obstruction such as lower extremity ischemia, kidney ischemia, treating varicose veins, deep vein thrombosis, hepatic artery chemoembolization, tumor emobilzation, uterine fibroids, etc.

The herein described subject matter sometimes illustrates different components or elements contained within, or connected with, different other components or elements. It is to be understood that such depicted architectures are merely examples, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality may be seen as “associated with” each other such that the desired functionality is achieved, irrespective of architectures or intermedial components. Likewise, any two components so associated may also be viewed as being “operably connected”, or “operably coupled”, to each other to achieve the desired functionality, and any two components capable of being so associated may also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

Although the invention herein has been described with respect to particular embodiments, it is understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed is:

1. An ultrasound diagnostic and therapeutic device comprising:
a catheter equipped with an intracorporeal ultrasound transducer at or near a distal end thereof, said intracorporeal ultrasound transducer is configured to emit and receive ultrasound signals for imaging purposes,
an extracorporeal ultrasound transmitter,
an imaging electronic unit connected to said intracorporeal ultrasound transducer, said imaging electronic unit is configured to generate an image of tissue adjacent to said intracorporeal ultrasound transducer, and
a time-reversal electronic unit configured to cause said extracorporeal ultrasound transmitter to send an ultrasound impulse towards said intracorporeal ultrasound transducer, said time-reversal electronic unit is further configured to receive an impulse response signal generated by said intracorporeal transducer upon receiving said ultrasound impulse at a first focusing point, said time-reversal electronic unit is further configured to cause said extracorporeal ultrasound transmitter to generate a high-intensity focused ultrasound area at a location of said first focusing point based on time-reversal of said impulse response signal.

2. The ultrasound diagnostic and therapeutic device as in claim 1, wherein said extracorporeal ultrasound transmitter comprises a reverberator and a plurality of ultrasound transducers operably coupled thereto.

3. The ultrasound diagnostic and therapeutic device as in claim 2, wherein said time-reversal electronic unit is further configured to cause said ultrasound transducers of said extracorporeal ultrasound transmitter to send said ultrasound impulses towards said intracorporeal ultrasound transducer one at a time, said time-reversal electronic unit is further configured to cause said extracorporeal ultrasound transmitter to generate said high-intensity focused ultrasound area by synchronously activating said plurality of ultrasound transducers all at the same time based on time-reversal of respective impulse response signals received one at a time by said intracorporeal ultrasound transducer.

4. The ultrasound diagnostic and therapeutic device as in claim 1, wherein said time-reversal electronic unit is configured to record a series of impulse response signals generated by said intracorporeal ultrasound transducer while at a plurality of focusing points along a trajectory of movement thereof, said time-reversal electronic unit is further configured to produce a focusing signal by superimposing at least some of said impulse response signals after time-reversing thereof, said at least some impulse response signals corresponding to selected focusing points of said plurality of focusing points, said time-reversal electronic unit is configured to cause said extracorporeal ultrasound transmitter to generate a composite high-intensity focused ultrasound area at said selected focusing points using said focusing signal.

5. The ultrasound diagnostic and therapeutic device as in claim 4, wherein said time-reversal electronic unit is further configured to record position data for each of said focusing points and pair thereof to a corresponding recorded impulse response signal.

6. The ultrasound diagnostic and therapeutic device as in claim 5, wherein said position data indicates locations of each of said focusing points relative to said first focusing point.

7. The ultrasound diagnostic and therapeutic device as in claim 1 further configured to identify a treatment site from at least one image generated by said imaging electronic unit when said intracorporeal ultrasound transducer is at said first focusing point.

8. The ultrasound diagnostic and therapeutic device as in claim 7 wherein said time-reversal focusing unit is further configured to cause said extracorporeal transmitter to generate said composite high-intensity focused ultrasound area shaped to correspond to said treatment site.

9. The ultrasound diagnostic and therapeutic device as in claim 4, wherein said time-reversal electronic unit is further configured to calculate an impulse response signal at at least one focusing point adjacent to said plurality of focusing points where said impulse response signal is recorded.

10. The ultrasound diagnostic and therapeutic device as in claim 9, wherein said time-reversal electronic unit is further configured to cause said extracorporeal transmitter to generate said composite high-intensity focused ultrasound area shaped to correspond to said treatment site using at least some of said focusing points where said impulse response signal was recorded or calculated.

11. The ultrasound diagnostic and therapeutic device as in claim 1, wherein said time-reversal electronic unit contains a library of prerecorded impulse response signals and their locations relative to said extracorporeal ultrasound transducer, said time-reversal electronic unit is further configured to select one of said prerecorded impulse response signals from said library to most closely correlate to said impulse response signal recorded at said first focusing point, said time-reversal electronic unit is further configured to select other prerecorded impulse response signals at locations corresponding to said treatment site, said time-reversal electronic unit is further configured to generate said focusing signal by superimposing all said selected impulse response signals so as to cause said extracorporeal transmitter to generate said composite high-intensity focused ultrasound area shaped to correspond to said treatment site using said focusing signal.

12. The ultrasound diagnostic and therapeutic device as in claim 11, wherein said library is obtained by measuring a plurality of impulse response signals and respective coordinates at various locations relative to said extracorporeal ultrasound transducer when said extracorporeal ultrasound transducer is placed in contact with a phantom fluid, said phantom fluid is characterized by ultrasound propagation speed being similar to that of soft tissues.

13. The ultrasound diagnostic and therapeutic device as in claim 12, wherein said phantom fluid is water or saline solution.
